(19) United States
(12) Patent Application Publication
(10) Pub. No.: US 2025/0073207 A1
(43) Pub. Date: Mar. 6, 2025

(54) PSILOCIN BENZOATE FORMULATION FOR INTRAVENOUS INFUSION

(71) Applicant: Eleusis Therapeutics US, Inc., New York, NY (US)

(72) Inventors: Alexander SCHWARZ, Baltimore, MD (US); David E. NICHOLS, Chapel Hill, NC (US); Claire WOMBWELL, Cambridge (GB); David Anthony FLEET, Northumberland (GB)

(21) Appl. No.: 18/728,208

(22) PCT Filed: Jan. 12, 2023
(86) PCT No.: PCT/US2023/010661

§ 371 Date: Jul. 11, 2024

Related U.S. Application Data
(60) Provisional application No. 63/298,839, filed on Jan. 12, 2022.

(51) Int. Cl. A61K 31/4045; A61K 9/00; A61K 9/08; A61K 31/407; A61K 31/5513; A61K 47/02; A61K 47/12; A61P 25/24

(52) U.S. Cl. CPC A61K 31/4045; A61K 9/0019; A61K 9/08; A61K 31/407; A61K 31/5513; A61K 47/02; A61K 47/12; A61P 25/24

(57) ABSTRACT

The invention features psilocin formulations for intravenous infusions including psilocin benzoate salt in combination with an antioxidant.

### BACKGROUND

Significant interest in the therapeutic application of psilocin has developed, based upon evidence of possible therapeutic effects in a wide array of clinical applications, including psychiatric conditions, pain disorders, and neurological conditions. However, due to the physical properties of psilocin, susceptibility to auto catalyzed oxidation upon handling and prolonged storage, and low water solubility, there exists a need for psilocin formulations for intravenous administration with improved stability.

### SUMMARY OF THE INVENTION

In a first aspect, the disclosure provides a pharmaceutical composition including (i) an aqueous solution having a pH of between about 3.5 and about 6.5 (e.g., 3.5±0.5, 4.0±0.5, 4.5±0.5, 5.0±0.5, 5.5±0.5, 6.0±0.5, and 6.5±0.5), (ii) between about 0.0053 mg/mL and about 4.0 mg/ml (e.g., 0.006±0.001 mg/mL, 0.0075±0.0025 mg/mL, 0.01±0.005 mg/mL, 0.05±0.025 mg/mL, 0.075±0.025 mg/mL, 0.1±0.05 mg/mL, 0.2±0.1 mg/mL, 0.3±0.1 mg/mL, 0.4±0.1 mg/mL, 0.5±0.1 mg/mL, 0.6±0.1 mg/mL, 0.7±0.1 mg/mL, 0.8±0.1 mg/mL, 0.9±0.1 mg/mL, 1±0.5 mg/mL, 2±1 mg/mL, 3±1 mg/mL, 4±1 mg/mL) of psilocin benzoate, and (iii) an antioxidant, wherein the pharmaceutical composition is suitable for infusion. In some embodiments, the aqueous solution includes a citrate buffer, an acetate buffer, or a phosphate buffer, and has a pH of between about 4.0 and about 6.0 (e.g., 4.0±0.5, 4.5±0.5, 5.0±0.5, 5.5±0.5, and 6.0±0.5).

In another aspect, the invention provides a pharmaceutical composition including (i) a citrate buffered aqueous solution having a pH of between about 4.0 and about 5.0 (e.g., 4.0±0.5, 4.5±0.5, and 5.0±0.5), and (ii) between about 0.0053 mg/ml and about 4.0 mg/ml (e.g., 0.006±0.001 mg/mL, 0.0075±0.0025 mg/mL, 0.01±0.005 mg/mL, 0.05±0.025 mg/mL, 0.075±0.025 mg/mL, 0.1±0.05 mg/mL, 0.2±0.1 mg/mL, 0.3±0.1 mg/mL, 0.4±0.1 mg/mL, 0.5±0.1 mg/mL, 0.6±0.1 mg/mL, 0.7±0.1 mg/mL, 0.8±0.1 mg/mL, 0.9±0.1 mg/mL, 1±0.5 mg/mL, 2±1 mg/mL, 3±1 mg/mL, 4±1 mg/mL) of psilocin benzoate, wherein the pharmaceutical composition is suitable for infusion.

In certain embodiments, the aqueous solution includes between about 0.001% (w/v) to 2% (w/v) of an antioxidant (e.g., 0.004±0.002 (w/v), 0.006±0.002% (w/v), 0.008±0.002% (w/v), 0.01±0.005% (w/v), 0.015±0.05% (w/v), 0.02±0.01% (w/v), 0.03±0.01% (w/v), 0.04±0.01% (w/v), 0.05±0.01% (w/v), 0.06±0.01% (w/v), 0.07±0.01% (w/v), 0.08±0.01% (w/v), 0.09±0.01% (w/v), 0.1±0.05% (w/v), 0.15±0.05% (w/v), 0.2±0.1% (w/v), 0.3±0.1% (w/v), 0.4±0.2% (w/v), 0.6±0.2% (w/v), 0.8±0.2% (w/v), 1±0.5% (w/V), 1.5±0.5% (w/v), and 2±0.5% (w/v) of an antioxidant). For example, the antioxidant may be in an amount of between 0.01% (w/v) and 2% (w/v) (e.g., 0.01±0.01% (w/v), 0.02±0.01% (w/v), 0.03±0.01% (w/V), 0.04±0.01% (w/v), 0.05±0.01% (w/v), 0.06±0.01% (w/v), 0.07±0.01% (w/v), 0.08±0.01% (w/V), 0.09±0.01% (w/v), 0.1±0.1% (w/v), 0.2±0.1% (w/v), 0.3±0.1% (w/v), 0.4±0.1% (w/v), 0.5±0.1% (w/V), 0.6±0.1% (w/v), 0.7±0.1% (w/v), 0.8±0.1% (w/v), 0.9±0.1% (w/v), 1±0.1% (w/v), 1.1±0.1% (w/v), 1.2±0.1% (w/v), 1.3±0.1% (w/v), 1.4±0.1% (w/v), 1.5±0.1% (w/v), 1.6±0.1% (w/v), 1.7±0.1% (w/v), 1.8±0.1% (w/v), 1.9±0.1% (w/v), and 2±0.1% (w/v)).

In some embodiments, the antioxidant is vitamin C, thioglycerol, sodium bisulfite, or sodium sulfite. In particular embodiments, the aqueous solution includes about 0.01±0.005% sodium bisulfite.

The pharmaceutical composition may further include one or more pharmaceutically acceptable excipients selected from a preservative, or a tonicity agent. In some embodiments, the pharmaceutical composition further includes from 0.1% (w/v) to 1% (w/v) (e.g., 0.2±0.1% (w/v), 0.3±0.1% (w/v), 0.4±0.1% (w/V), 0.5±0.1% (w/v), 0.6±0.1% (w/v), 0.7±0.1% (w/v), 0.8±0.1% (w/v), 0.9±0.1% (w/v) and 1±0.1% (w/v)) sodium chloride as a tonicity agent.

In particular embodiments, the pharmaceutical composition includes: (i) a citrate buffered aqueous solution having a pH of between 4.0 and 5.0 (e.g., 4.0±0.5, 4.5±0.5, and 5.0±0.5), (ii) between about 0.0053 mg/mL and about 0.7 mg/ml (e.g., 0.006±0.001 mg/mL, 0.0075±0.0025 mg/mL, 0.01±0.005 mg/mL, 0.05±0.025 mg/mL, 0.075±0.025 mg/mL, 0.1±0.05 mg/mL, 0.2±0.1 mg/mL, 0.3±0.1 mg/mL, 0.4±0.05 mg/mL, 0.45±0.05 mg/mL, 0.5±0.05 mg/mL, 0.55±0.05 mg/mL, 0.6±0.05 mg/mL, 0.65±0.05 mg/mL, and 0.7±0.05 mg/mL) of psilocin benzoate, and (iii) 0.01 to 0.075% (w/v) sodium bisulfite (e.g., 0.02±0.01% (w/v), 0.03±0.01% (w/v), 0.4±0.01% (w/v), 0.05±0.01% (w/v), 0.06±0.01% (w/v), and 0.07±0.01% (w/v) sodium bisulfite).

In some embodiments, the pharmaceutical composition includes from about 50 to about 200 mM citrate buffer (e.g., 50±10 mM, 60±10 mM, 70±10 mM, 80±10 mM, 90±10 mM, 100±10 mM, 110±10 mM, 120±10 mM, 130±10 mM, 140±10 mM, 150±10 mM, 160±10 mM, 170±10 mM, 180±10 mM, 190±10 mM, and 200±10 mM).

In another aspect, the disclosure provides a reconstitutable powder including psilocin benzoate, an antioxidant, and a buffer, wherein reconstitution of the reconstitutable powder in from 5 mL to 50 mL (e.g., 5±1 mL, 6±1 mL, 7±1 mL, 8±1 mL, 9±1 mL, 10±5 mL, 15±5 mL, 20±5 mL, 25±5 mL, 30±5 mL, 35±5 mL, 40±5 mL, 45±5 mL, and 50±5 mL) of an aqueous solution produces any one of the pharmaceutical compositions described herein. In certain embodiments, the powder includes between about 0.01% (w/w) and 2% (w/w) of psilocin benzoate (e.g., 0.01±0.01% (w/w), 0.02±0.01% (w/w), 0.03±0.01% (w/w), 0.04±0.01% (w/w), 0.05±0.01% (w/w), 0.06±0.01% (w/w), 0.07±0.01% (w/w), 0.08±0.01% (w/w), 0.09±0.01% (w/w), 0.1±0.1% (w/w), 0.2±0.1% (w/w), 0.3±0.1% (w/w), 0.4±0.1% (w/w), 0.5±0.1% (w/w), 0.6±0.1% (w/w), 0.7±0.1% (w/w), 0.8±0.1% (w/w), 0.9±0.1% (w/w), 1±0.1% (w/w), 1.1±0.1% (w/w), 1.2±0.1% (w/w), 1.3±0.1% (w/w), 1.4±0.1% (w/w), 1.5±0.1% (w/w), 1.6±0.1% (w/w), 1.7±0.1% (w/w), 1.8+0.1% (w/w), 1.9+0.1% (w/w), and 2+0.1% (w/w)).

In another aspect, the disclosure provides a method of treating a disease or condition in a subject in need thereof including intravenously administering to the subject any one of the pharmaceutical compositions described herein in an amount sufficient to treat the disease or condition. In some embodiments, the method includes intravenously administering to the subject any one of the pharmaceutical compositions described herein over a period of between 1 minute and 60 minutes (e.g., between 1 minute and 10 minutes, 10 minutes and 60 minutes, 20 minutes and 60 minutes, 30 minutes and 60 minutes, 40 minutes and 60 minutes, 50 minute and 60 minutes, 1 minute and 10 minutes, 1 minute and 20 minutes, 1 minute and 30 minutes, 1 minutes and 40 minutes, and 1 minute and 50 minutes). For example, the pharmaceutical composition may be administered to the subject over a period of 20 to 60 minutes (e.g., 20 minutes to 50 minutes, 20 minutes to 40 minutes, 20 minutes to 30 minutes, 30 minutes to 60 minutes, 40 minutes to 60 minutes, and 50 minutes and 60 minutes.

In some embodiments, the disease or condition is a neurological injury, a neurodegenerative disease, an inflammatory condition, chronic pain, or a psychological condition. In particular embodiments, the disease or condition is an inflammatory condition. In certain embodiments, the inflammatory condition is lung inflammation, neuroinflammation, rheumatoid arthritis, atherosclerosis, psoriasis, type II diabetes, inflammatory bowel disease, Crohn's disease, multiple sclerosis, and/or septicemia. In some embodiments, the inflammatory condition is chronic obstructive pulmonary disease (COPD), or Alzheimer's disease. In certain embodiments, the disease or condition is a neurological injury. In particular embodiments, the neurological injury is a stroke, a traumatic brain injury, or a spinal cord injury.

In some embodiments, the disease or condition is chronic pain. The chronic pain may result from post-operative pain, tension headaches, chronic lower back pain, fibromyalgia, nephropathy, multiple sclerosis, shingles, complex regional pain syndrome, cephalic pain, or sciatica. In particular embodiments, the chronic pain condition results from trigeminal autonomic cephalalgia. The trigeminal autonomic cephalalgia may be selected from the group consisting of episodic and chronic cluster headache (CH), episodic and chronic paroxysmal hemicrania (PH), and short-lasting unilateral neuralgiform headache attacks with conjunctival injection and tearing (SUNCT). In particular embodiments, the trigeminal autonomic cephalalgia is episodic or chronic CH.

In some embodiments, the condition is a psychological condition. The psychological condition may be depression, anxiety, addiction, post-traumatic stress disorder, an eating disorder, or compulsive behavior. For example, the psychological condition may be depression. In particular embodiments, the psychological condition is anxiety.

In certain embodiments the disease or condition is a neurodegenerative disease selected from Alzheimer's disease, Huntington's disease, and Parkinson's disease.

In certain embodiments, the method includes further administering to the patient a pharmacologically effective amount of an antiemetic agent. The antiemetic agent may include a non- selective 5-HT antagonist, 5-HT3 receptor antagonist, 5-HT4 receptor agonist, CB1 agonist, D2 receptor antagonist, D3 receptor antagonist, GABA receptor agonist, H1 receptor antagonist, muscarinic acetylcholine receptor antagonist, NK1 receptor antagonist, or a combination thereof. In particular embodiments, the antiemetic ondansetron is intravenously infused.

In some embodiments, the intravenous infusion includes a pharmacologically effective amount of a benzodiazepine. The benzodiazepine may be a 1,4-benzodiazepine, 1,5-benzodiazepine, 2,3-benzodiazepine, triazolobenzodiazepine, imidazobenzodiazepine, oxazolobenzodiazepine, thienodiazepine, thienotriazolodiazepine, thienobenzodiazepine,pyridodiazepine, pyridotriazolodiazepine, pyrralodiazepine, tetrahydroisoquinobenzodiazepine, a benzodiazepine prodrug, or a combination thereof. In particular embodiments, the benzodiazepine is lorazepam or diazepam. In some embodiments, the benzodiazepine is administered in a dosage between 2 mg and 10 mg (e.g., 2±1 mg, 3±1 mg, 4±1 mg, 5±1 mg, 6±1 mg, 7±1 mg, 8±1 mg, 9±1 mg, and 10±1 mg).

In some embodiments, the intravenous infusion includes a pharmacologically effective amount of an anesthetic, a sedative, an antiemetic, an anticonvulsant, an antidepressant, an antimigraine, an antipsychotic, an anxiolytic, and/or an antiparkinson agent.

In some embodiments, the method further includes administering to the patient a preparation including a pharmacologically effective amount of an anti-inflammatory agent. In certain embodiments, the administration of the preparation is an intravenous infusion of ketorolac or pharmaceutically acceptable salt thereof. In certain embodiments, the administration of the preparation is an intramuscular infusion of a pharmacologically effective amount of a triptan or a pharmaceutically acceptable salt thereof. In particular embodiments, the preparation includes sumatriptan or a pharmaceutically acceptable salt thereof.

In some embodiments, the intravenous infusion including a pharmacologically effective amount of psilocin benzoate is administered at least twice over the course of a month (e.g., at least two times, three times, four times, five times, six times, seven times, eight times, nine times, ten times, or more over the course of a month). In some embodiments, the intravenous infusion including a pharmacologically effective amount of psilocin benzoate is administered between 2 and 10 times over the course of a year (e.g., 2 times, 3 times, 4 times, 5 times, 6 times, 7 times, 8 times, 9 times, or 10 times over the course of a year).

### Definitions

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

Terms such as “a”, “an,” and “the” are not intended to refer to only a singular entity but include the general class of which a specific example may be used for illustration.

As used herein, the term “about” refers to a value that is within 10% above or below the value being described.

As used herein, the terms “acute stress disorder” and “ASD” refer to a condition that arises as a response to a stressful event or situation of an exceptionally threatening or catastrophic nature, which is likely to cause pervasive distress in an individual (e.g., natural or man-made disaster, combat, serious accident, witnessing the violent death of others, or being the victim of torture, terrorism, rape, or other crime). Like PTSD, acute stress disorder is an anxiety disorder that involves a very specific reaction following exposure to a traumatic event or stressor. However, the duration of acute stress disorder is shorter than that for PTSD, such that the symptoms are present for at least one, two, or three days, but no more than four, five, or six weeks. For individuals exhibiting symptoms persisting for a longer period of time, a diagnosis of PTSD may be warranted.

The term “administration” or “administering” refers to a method of giving a dosage of a compound or pharmaceutical composition to a subject.

As used herein, the term “continuous infusion” refers to an infusion of a drug (e.g., psilocin benzoate) such that the plasma concentration of the drug and/or metabolite (e.g., psilocin) does not vary by more than ±10% for at least 15 minutes, unless the rate of infusion is altered in response to the subject's intensity rating.

By “dysthymia” or “dysthymic disorder” is meant a chronically depressed mood that occurs for most of the day, more days than not, for at least two years. In children and adolescents, the mood may be irritable rather than depressed, and the required minimum duration is one year. During the two-year period (one year for children or adolescents), any symptom-free intervals last no longer than 2 months. During periods of depressed mood, at least two of the following additional symptoms are present: poor appetite or overeating, insomnia or hypersomnia, low energy or fatigue, low self-esteem, poor concentration, or difficulty making decisions, and feelings of hopelessness. The symptoms cause clinically significant distress or impairment in social, occupational (or academic), or other important areas of functioning. The diagnosis of dysthymia is not made if: the individual has ever had a manic episode, a mixed episode, a hypomanic episode; has ever met the criteria for a cyclothymic disorder; the depressive symptoms occur exclusively during the course of a chronic psychotic disorder (e.g., schizophrenia); or if the disturbance is due to the direct physiological effects of a substance or a general medical condition. After the initial two-years of dysthymic disorder, major depressive episodes may be superimposed on the dysthymic disorder (“double depression”). Diagnostic and Statistical Manual of Mental Disorders (OSM IV), American Psychiatric Press, 4th Edition, 1 994. Diagnostic guidance for psychological disorders can be found, for example, in the ICD-10 (The ICD-10 Classification of Mental and Behavioral Disorders: Diagnostic Criteria for Research, Geneva: World Health Organization, 1993) and the DSM-V (American Psychiatric Association. Diagnostic and Statistical Manual of Mental Disorders, Fifth Edition (DSM-V) Arlington, VA.; American Psychiatric Association, 2013).

By “free base equivalent” is meant an amount corresponding to a free base equivalent in a mass of psilocin benzoate. For example, a free base equivalent of 1 mg of psilocin is equal to 1 mg of psilocin in its free base form and equal to 1.60 mg of psilocin in its benzoate salt form (e.g., 1.0×(326.39/204.27) to account for the mass contribution of the benzoic acid).

As used herein, the term “generalized anxiety disorder” refers to a condition characterized by excessive anxiety and worry (i.e., apprehensive expectation). Typically, the excessive anxiety and worry occur on more days than not for a period of time (e.g., one, two, three, or four months or more). The anxiety and worry can be associated with (i) restlessness, feeling keyed up, or on edge; and/or (ii) muscle tension. The anxiety and worry can be associated with (a) a marked avoidance of situations in which a negative outcome could occur; (b) a marked time and effort preparing for situations in which a negative outcome could occur; (c) a marked procrastination in behavior or decision-making due to worries; and (d) repeatedly seeking reassurance due to worries. The anxiety, worry, or physical symptoms can cause clinically significant distress or impairment in social, occupational, or other important areas of functioning in many, but not necessarily all individuals with GAD.

As used herein, the terms “obsessive compulsive disorder,” “OCD,” and “anxiety and obsessive- compulsive spectrum disorders” refer to a condition characterized by obsessions and/or compulsions. Obsessions are recurrent and persistent thoughts, urges, or images that are experienced, at some time during the disturbance, as intrusive and unwanted and that usually cause marked anxiety or distress in which the obsessed individual attempts to ignore or suppress such thoughts, urges, or images, or to neutralize them with some other thought or action (i.e., by performing a compulsion). Compulsions are repetitive behaviors (e.g., hand washing, ordering, checking) or mental acts (e.g., praying, counting, repeating words silently) that the person feels driven to perform in response to an obsession, or according to rules that must be applied rigidly. The behaviors or mental acts are aimed at preventing or reducing anxiety or distress, or preventing some dreaded event or situation; however, these behaviors or mental acts either are not connected in a realistic way with what they are designed to neutralize or prevent, or are clearly excessive. Typically the obsessions or compulsions are time consuming (for example, take more than 1 hour a day), or cause clinically significant distress or impairment in social, occupational, or other important areas of functioning.

As used herein, the term “panic disorder” refers to a condition characterized by recurrent and unexpected panic attacks. Panic disorder includes both panic disorder with agoraphobia and panic disorder without agoraphobia. Subjects with this condition can exhibit one or both of the following: (i) a persistent concern or worry about additional panic attacks or their consequences (e.g., losing control, having a heart attack, going crazy); and/or (ii) significant maladaptive change in behavior related to the attacks (e.g., behaviors designed to avoid having panic attacks), which may include agoraphobic avoidance.

As used herein, the terms “pharmacologically effective amount,” “therapeutically effective amount,” and the like, when used in reference to a therapeutic composition, refer to a quantity sufficient to, when administered to the subject, including a mammal, for example a human, effect beneficial or desired results, such as clinical results. For example, in the context of treating depression, described herein, these terms refer to an amount of the composition sufficient to achieve a treatment response as compared to the response obtained without administration of the composition. The quantity of a given composition described herein that will correspond to such an amount may vary depending upon various factors, such as the given agent, the pharmaceutical formulation, the route of administration, the type of disease or disorder, the identity of the subject (e.g., age, sex, weight) or host being treated, and the like. An “effective amount,” “pharmacologically effective amount,” or the like, of a composition of the present disclosure, also include an amount that results in a beneficial or desired result in a subject as compared to a control (e.g., a decrease in the score on the Montgomery-Asberg Depression Rating Scale).

As used herein, the terms “post traumatic stress disorder” and “PTSD” refer to a condition that arises as a delayed and/or protracted response to a stressful event or situation (either short-or long-lasting) of an exceptionally threatening or catastrophic nature, which is likely to cause pervasive distress in an individual (e.g., natural or man-made disaster, combat, serious accident, witnessing the violent death of others, or being the victim of torture, terrorism, rape, or other crime). Predisposing factors such as personality traits (e.g., compulsive, asthenic) or previous history of neurotic illness may lower the threshold for the development of the condition or aggravate its course, but they are neither necessary nor sufficient to explain its occurrence. PTSD is a less frequent and more enduring consequence of psychological trauma than the more frequently seen acute stress response. PTSD has been recognized in the past as railway spine, stress syndrome, shell shock, battle fatigue, traumatic war neurosis, and post-traumatic stress syndrome. Diagnostic symptoms include re-experiencing original trauma(s), by means of flashbacks or nightmares; avoidance of stimuli associated with the trauma; and increased arousal, such as difficulty falling or staying asleep, anger, and hypervigilance. Formal diagnostic criteria (DSM-V, DSM-IV, and/or ICD-9) require that the symptoms last more than one month and cause significant impairment in social, occupational, or other important areas of functioning (e.g., problems with work and/or relationships). Formal diagnostic criteria can include: (i) intrusion symptoms that are associated with the traumatic event (e.g., (a) spontaneous or cued recurrent, involuntary, and intrusive distressing memories of the traumatic event; (b) recurrent distressing dreams in which the content and/or affect of the dream is related to the event; (c) dissociative reactions (e.g., flashbacks) in which the individual feels or acts as if the traumatic event were recurring (such reactions may occur on a continuum, with the most extreme expression being a complete loss of awareness of present surroundings; (d) intense or prolonged psychological distress at exposure to internal or external cues that symbolize or resemble an aspect of the traumatic event; and/or (e) marked physiological reactions to reminders of the traumatic event); (ii) persistent avoidance of stimuli associated with the traumatic event (e.g., (a) thoughts, feelings, or physical sensations that arouse recollections of the traumatic event; (b) activities, places, physical reminders, or times (e.g., anniversary reactions) that arouse recollections of the traumatic event; and/or (c) people, conversations, or interpersonal situations that arouse recollections of the traumatic event); (iii) negative alterations in cognitions and mood that are associated with the traumatic event (e.g., (a) inability to remember an important aspect of the traumatic event (typically dissociative amnesia); (b) persistent and exaggerated negative expectations about one's self, others, or the world; (c) persistent distorted blame of self or others about the cause or consequences of the traumatic event; (d) pervasive negative emotional state (e.g., fear, horror, anger, guilt, or shame); (e) markedly diminished interest or participation in significant activities; (f) feeling of detachment or estrangement from others; and/or (g) persistent inability to experience positive emotions (e.g., unable to have loving feelings, psychic numbing); and (iv) alterations in arousal (i.e., hyperarousal) and reactivity that are associated with the traumatic event (e.g., (a) irritable, angry, or aggressive behavior; (b) reckless or self-destructive behavior; (c) hypervigilance; (d) exaggerated startle response; (e) problems with concentration; and/or (f) sleep disturbance (e.g., difficulty falling or staying asleep, or restless sleep)). Formal diagnostic criteria can further include that the duration of disturbance is more than a certain period of time (e.g., one month, three months, or six months) and that the disturbance causes clinically significant distress or impairment in social, occupational, or other important areas of functioning. In a small proportion of patients the condition may show a chronic course over many years and a transition to an enduring personality change. The three main symptoms associated with PTSD are (1) “reliving” the traumatic event, such as flashbacks, nightmares, intrusive thoughts and recollections, (2) avoidance behaviors and emotional numbing, and (3) hypersensitivity such as an inability to sleep, anxious feelings, overactive startle response, hyperarousal, hypervigilance, irritability, and outbursts of anger.

As used herein, the terms “psychological disorder” and “psychological condition” refer to a condition characterized by a disturbance in one's emotional or behavioral regulation that reflects a dysfunction in the psychological, biological, or developmental processes underlying mental function.

Psychological disorders include, but are not limited to depressive disorders (major depression, treatment resistant depression, melancholic depression, atypical depression, or dysthymia), anxiety disorders (end of life anxiety, generalized anxiety disorder, panic disorder, social anxiety, post-traumatic stress disorder, acute stress disorder, obsessive compulsive disorder, or social phobia), addictions (e.g., substance abuse, e.g., alcoholism, tobacco abuse, or drug abuse)), eating disorders (e.g., anorexia nervosa, bulimia nervosa, and binge eating disorder) and compulsive behavior disorders (e.g., primary impulse-control disorders or obsessive-compulsive disorder). Psychological disorders can be any psychological condition associated with one or more symptoms, e.g., somatic symptoms (e.g., chronic pain, anxiety disproportionate to severity of physical complaints, pain disorder, body dysmorphia, conversion (i.e., loss of bodily function due to anxiety), hysteria, or neurological conditions without identifiable cause), or psychosomatic symptoms (e.g., back pain, fibromyalgia, migraines, and chronic fatigue syndrome). Psychological disorders also include repetitive body-focused behaviors, such as tic disorders (e.g., Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder). In some cases, development of a psychological disorder is associated with or characterized by a prodromal symptom, such as depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, and decreased initiation of voluntary responses.

As used herein, the terms “social phobia” and “social anxiety disorder” refer to a condition characterized by fear or anxiety associated with one or more social situations. Subjects with this condition typically exhibit a marked fear or anxiety about one or more social situations in which the person is exposed to possible scrutiny by others. Examples include social interactions (e.g., having a conversation), being observed (e.g., eating or drinking), or performance in front of others (e.g., giving a speech). Typically, an individual with this condition (i) fears that he or she will act in a way, or show anxiety symptoms that will be negatively evaluated (i.e., be humiliating, embarrassing, lead to rejection, or offend others); (ii) the social situations almost invariably provoke immediate fear or anxiety; (iii) the social situations are avoided or endured with intense fear or anxiety; and (iv) the fear or anxiety is out of proportion to the danger posed by the social situation. In children, the fear or anxiety may be expressed by crying, tantrums, freezing, clinging, shrinking or refusal to speak in social situations. The fear, anxiety, and avoidance can cause clinically significant distress or impairment in social, occupational, or other important areas of functioning.

As used herein, the terms “treat,” “treating,” or “treatment” refer to administration of a compound or pharmaceutical composition for a therapeutic purpose. To “treat a disorder” or use for “therapeutic treatment” refers to administering treatment to a patient already suffering from a disease to ameliorate the disease or one or more symptoms thereof to improve the patient's condition (e.g., by reducing one or more symptoms of inflammation). The term “therapeutic” includes the effect of mitigating deleterious clinical effects of certain inflammatory processes (i.e., consequences of the inflammation, rather than the symptoms of inflammation). The methods of the invention can be used as a primary prevention measure, i.e., to prevent a condition or to reduce the risk of developing a condition. Prevention refers to prophylactic treatment of a patient who may not have fully developed a condition or disorder, but who is susceptible to, or otherwise at risk of, the condition. Thus, in the claims and embodiments, the methods of the invention can be used either for therapeutic or prophylactic purposes.

By “major depressive disorder” is meant a clinical course that is characterized by one or more major depressive episodes in an individual without a history of manic, mixed, or hypomanic episodes. The diagnosis of unipolar depression is not made if: manic, mixed, or hypomanic episodes develop during the course of depression; if the depression is due to the direct physiological effects of a substance; if the depression is due to the direct physiological effects of a general medical condition; if the depression is due to a bereavement or other significant loss (“reactive depression”); or if the episodes are better accounted for by schizoaffective disorder and are not superimposed on schizophrenia, schizophreniform disorder, delusional disorder, or psychotic disorder. If manic, mixed, or hypomanic episodes develop, then the diagnosis is changed to a bipolar disorder. Depression may be associated with chronic general medical conditions (e.g., diabetes, myocardial infarction, carcinoma, and stroke). Generally, unipolar depression is more severe than dysthymia. The essential feature of a major depressive episode is a period of at least two to 15 weeks during which there is either depressed mood or loss of interest or pleasure in nearly all activities. In children and adolescents, the mood may be irritable rather than sad. The episode may be a single episode or may be recurrent. The individual also experiences at least four additional symptoms drawn from a list that includes changes in appetite or weight, sleep, and psychomotor activity; decreased energy; feelings of worthlessness or guilt; difficulty thinking, concentrating, or making decisions; or recurrent thoughts of death or suicidal ideation, plans, or attempts. Each symptom must be newly present or must have clearly worsened compared with the person's pre-episode status. The symptoms must persist for most of the day, nearly every day, for at least two consecutive weeks, and the episode must be accompanied by clinically significant distress or impairment in social, occupational (or academic), or other important areas of functioning (Diagnostic and Statistical Manual of Mental Disorders (OSM IV), American Psychiatric Press, 4th Edition, 1994). Diagnostic guidance for psychological disorders can be found, for example, in the ICD-10 (The ICD-10 Classification of Mental and Behavioral Disorders: Diagnostic Criteria for Research, Geneva: World Health Organization, 1993) and the DSM-V (American Psychiatric Association. Diagnostic and Statistical Manual of Mental Disorders, Fifth Edition (DSM-V) Arlington, VA.; American Psychiatric Association, 2013).

Other features and advantages of the invention will be apparent from the following Detailed Description, Examples, and Claims.

### DETAILED DESCRIPTION OF THE INVENTION

The disclosure provides new psilocin formulations including psilocin benzoate salt in combination with an antioxidant. The psilocin benzoate formulations described herein are suitable for intravenous infusion. The invention further features methods of treating psychological conditions, neurological injuries, pain, cephalic pain (e.g., headache), inflammatory conditions, and anxiety in a subject by utilizing the intravenous psilocin benzoate infusion formulations. The psilocin benzoate formulations may be administered in combination with another therapeutic agent, such as an antiemetic, a benzodiazepine, and/or an anti-inflammatory agent.

### Psilocin

Psilocin has the structure:

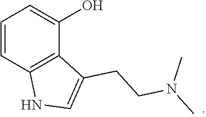

Psilocybin is a phosphate prodrug for psilocin. Psilocin is much more lipid soluble in comparison to psilocybin, and therefore is capable of crossing the blood brain barrier more effectively to elicit a response. Psilocin has a high affinity for and is able to activate the 5-HT2A receptor, which plays a key role in regulating mood, sexual behavior, aggression, impulsivity, cognitive function, appetite, pain, sleep, and memory along with other behaviors. As result, psilocin has effects at the 5-HT2A receptor that mimic the action of the endogenous neurotransmitter serotonin. This disclosure provides methods for intravenous administration of psilocin benzoate that are useful in therapy, such as in the treatment of a patient having a psychological condition or a neurological injury. The psilocin benzoate described herein may be a 1:1 benzoate salt.

### Compositions

The invention features pharmaceutical compositions for infusion of psilocin benzoate, including a buffer and/or an antioxidant. The pharmaceutical compositions described herein may include one or more pharmaceutically acceptable excipients. Examples of pharmaceutically acceptable excipients include, but are not limited to, biocompatible vehicles, adjuvants, additives, and diluents to achieve a composition usable as a dosage form.

The pharmaceutical compositions of the invention can include one or more solvents, diluents, or other liquid vehicle, surface active agents, tonicity agents, and/or preservatives, as suited to the particular dosage form desired. Remington's Pharmaceutical Sciences, 23^{rd} Edition, E. W. Martin (Mack Publishing Co., Easton, Pa., 1990) discloses various excipients used in formulating pharmaceutical compositions and known techniques for the preparation thereof. Some examples of materials that can serve as pharmaceutically acceptable excipients include, but are not limited to, sugars such as lactose, glucose and sucrose; cellulose and its derivatives such as sodium carboxymethyl cellulose, ethyl cellulose and cellulose acetate; buffering agents such as citrate, acetate, and/or phosphate; pyrogen-free water; isotonic saline; Ringer's solution; 5% dextrose solution and combinations with the foregoing aqueous solutions. For example, the pharmaceutical composition may include between 50 mM and 200 mM (e.g., 50±10 mM, 60±10 mM, 70±10 mM, 80±10 mM, 90±10 mM, 100±10 mM, 110±10 mM, 120±10 mM, 130±10 mM, 140±10 mM, 150±10 mM, 160±10 mM, 170±10 mM, 180±10 mM, 190±10 mM, and 200±10 mM) of a buffering agent.

The pharmaceutical compositions of the invention may be in the form of a powder that can be reconstituted (e.g., a lyophilized powder). In some embodiments, the composition can be stored as a lyophilized powder that is reconstituted prior to administration to a subject. The lyophilized powder may be reconstituted prior to administration in, e.g., water, normal saline, Ringer-Lactate solution, Ringer solution, or a buffered aqueous solution. The lyophilized powder can provide a longer stable shelf-life for the psilocin benzoate drug product.

The above-described compositions, in any of the forms described above, can be used for treating a disease or condition described herein. An effective amount refers to the amount of an active compound/agent that is required to confer a therapeutic effect on a treated subject. Effective doses will vary, as recognized by those skilled in the art, depending on the types of diseases treated, route of administration, excipient usage, and the possibility of co-usage with other therapeutic treatment. A pharmaceutical composition of this invention can be administered parenterally or intravenously. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

For use in the methods and compositions of the invention, the pharmaceutically acceptable psilocin salt may be contained in any appropriate amount in any suitable carrier substance formulated for intravenous infusion. Compositions for infusion use may be provided in unit dosage forms (e.g., in single-dose ampoules), or in vials containing several doses and in which a suitable preservative may be added. The solution of the pharmaceutically acceptable psilocin salt suitable for intravenous infusion may have a pH of about 3 to about 6 (e.g., 3±1, 4±1, 5±1, or 6±1). Furthermore, the solution of the pharmaceutically acceptable psilocin salt suitable for intravenous infusion may include a concentration of the pharmaceutically acceptable psilocin benzoate salt between about 0.0053 mg/mL and about 5.0 mg/ml (e.g., 0.006±0.001 mg/mL, 0.0075±0.0025 mg/mL, 0.01±0.005 mg/mL, 0.05±0.025 mg/mL, 0.075±0.025 mg/mL, 0.1±0.05 mg/mL, 0.2±0.1 mg/mL, 0.3±0.1 mg/mL, 0.4±0.1 mg/mL, 0.5±0.5 mg/mL, 1±0.5 mg/mL, 2±1 mg/mL, 3±1 mg/mL, 4±1 mg/mL, and 5.5±0.5 mg/mL). For example, the solution of the pharmaceutically acceptable psilocin salt suitable for intravenous infusion may include a concentration of the pharmaceutically acceptable psilocin benzoate salt between about 0.0053 mg/ml and about 0.7 mg/ml (e.g., 0.006±0.001 mg/mL, 0.0075±0.0025 mg/mL, 0.01±0.005 mg/mL, 0.05±0.025 mg/mL, 0.075±0.025 mg/mL, 0.1±0.05 mg/mL, 0.15±0.05 mg/mL, 0.2±0.05 mg/mL, 0.25±0.05 mg/mL 0.3±0.1 mg/mL, 0.35±0.05 mg/mL, 0.4±0.1 mg/mL, 0.45±0.05 mg/mL, 0.5±0.05 mg/mL, 0.55±0.05 mg/mL, 0.6±0.05 mg/mL, 0.65±0.05 mg/mL, and 0.7±0.05 mg/mL).

The pharmaceutical compositions of the invention include an antioxidant. An antioxidant may be used to improve the shelf-life stability of the psilocin benzoate. Antioxidants are known in the art. Non-limiting examples of an antioxidant include pyrroloquinoline quinone (PQQ), vitamin C, vitamin E (e.g., a tocopherol, such as a-tocopherol, B-tocopherol, or y-tocopherol, or a mixture thereof), B-carotene, a polyphenol (e.g., a phenolic acid, a stilbene, or a flavonoid), or an inorganic selenium. In some embodiments, the antioxidant is selected from the group consisting of pyrroloquinoline quinone (PQQ), vitamin C, vitamin E, B-carotene, a polyphenol, thioglycerol, sodium bisulfite, carnosine, N-acetylcarnosine, pyruvate, astaxanthin, glutathione, cysteine, cysteine and combinations thereof. The composition of the invention may be formulated to provide the antioxidant in an amount of between about 0.001% (w/v) to 5% (w/v). For example, the antioxidant may be in an amount of between 0.01% (w/v) and 2% (w/v) (e.g., 0.01±0.01% (w/v), 0.02±0.01% (w/v), 0.03±0.01% (w/v), 0.04±0.01% (w/v), 0.05±0.01% (w/v), 0.06±0.01% (w/v), 0.07±0.01% (w/v), 0.08±0.01% (w/v), 0.09±0.01% (w/v), 0.1±0.1% (w/v), 0.2±0.1% (w/v), 0.3±0.1% (w/v), 0.4±0.1% (w/v), 0.5±0.1% (w/v), 0.6±0.1% (w/v), 0.7±0.1% (w/v), 0.8±0.1% (w/v), 0.9±0.1% (w/v), 1±0.1% (w/v), 1.1±0.1% (w/v), 1.2±0.1% (w/v), 1.3±0.1% (w/v), 1.4±0.1% (w/v), 1.5±0.1% (w/v), 1.6±0.1% (w/v), 1.7±0.1% (w/v), 1.8±0.1% (w/v), 1.9±0.1% (w/v), and 2±0.1% (w/v)).

A sterile injectable composition can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils are conventionally employed as a solvent or suspending medium (e.g., synthetic mono-or diglycerides). Fatty acids, such as, but not limited to, oleic acid and its glyceride derivatives, are useful in the preparation of injectables, as are natural pharmaceutically acceptable oils. Other commonly used surfactants, such as, but not limited to, Tweens or Spans, which are commonly used in the manufacture of pharmaceutically acceptable liquid dosage forms also can be used for the purpose of formulation.

The above-described compositions, in any of the forms described above, may be stored in a light impenetrable container. For the example, the compositions described herein may be contained in an amber bottle or vial.

### Treatment Methods

The disclosure provides intravenous psilocin benzoate formulations useful for treating psychological conditions, neurological injuries, pain, cephalic pain (e.g., headache), inflammatory conditions, and anxiety.

### Psychological Conditions

The psilocin benzoate formulations of the invention can be used to treat psychological conditions. The psychological condition may be any psychological condition described herein. In some embodiments the psychological condition is depression, anxiety, addiction, post-traumatic stress disorder (PTSD), an eating disorder, or compulsive behavior. In some embodiments, the psychological condition may be depression. The psychological condition may also be anxiety. The anxiety may be experienced by a subject who is receiving palliative care or is enrolled in a hospice program. In certain embodiments, the subject who is experiencing anxiety has symptoms such as hypervigilance, fatigue, racing thoughts, irritability, excessive worry, and/or fear.

The subject diagnosed with a psychological condition may be diagnosed by evaluation of the subject's symptoms by a physician, clinician, or therapist based on a physical examination. For example, a blood test may be used to evaluate blood concentration levels of certain biomarkers such as hormones, calcium, vitamin D, electrolytes, and iron in diagnosing depression. Additionally, or alternatively, for patients with a possible depression condition a depression screening test may be performed by the physician, clinician, or therapist to aid in the diagnosis of depression. In some embodiments, the methods described herein may be used to treat psychosomatic pain conditions. In some embodiments, the psychosomatic pain condition may be fibromyalgia, chronic fatigue, migraines, or back pain.

### Neurological Injuries

The psilocin benzoate formulations of the invention can be used to treat a neurological injury. The neurological injury may be any neurological injury. In some embodiments, the neurological injury is a stroke, a traumatic brain injury, or a spinal cord injury. The methods of treating a neurological injury described herein may reduce acute inflammation. In certain embodiments, hippocampal hyperactivity is reduced. In particular embodiments, the methods of the invention are used to treat a neurological injury, e.g., stroke, traumatic brain injury, and spinal cord injury, by administering intravenous infusions of psilocin benzoate with an antioxidant as needed for pain, inflammation, and/or other symptoms associated with the neurological injury.

### Neurodegenerative Conditions

The psilocin benzoate formulations of the invention can be used to treat neurodegenerative conditions. The neurodegenerative condition to be treated can be Alzheimer's disease, Huntington's disease, or Parkinson's disease, among others.

### Inflammatory Conditions

The psilocin benzoate formulations of the invention can be used to treat inflammatory conditions. The inflammatory condition to be treated can be a lung inflammation (e.g., chronic obstructive pulmonary disease (COPD)), neuroinflammation (e.g., inflammation associated with Alzheimer's disease), chronic inflammation, rheumatoid arthritis, atherosclerosis, psoriasis, type II diabetes, inflammatory bowel disease, Crohn's disease, multiple sclerosis, and/or septicemia.

### Chronic Pain

The psilocin benzoate formulations of the invention can be used to treat conditions associated with chronic pain. The chronic pain may result from post-operative pain, tension headaches, chronic lower back pain, fibromyalgia, nephropathy, multiple sclerosis, shingles, complex regional pain syndrome, cephalic pain, or sciatica. The chronic pain may arise from an operation. The chronic pain may also be pain associated with a particular disease or condition such as nephropathy, multiple sclerosis, shingles, or complex regional pain syndrome. As used herein, a disorder or condition associated with cephalic pain is a disorder or condition which has as one of its symptoms cephalic/head pain (e.g., headache). Examples of such disorders or conditions include trigeminal autonomic cephalalgias such as episodic and chronic cluster headache (CH), episodic and chronic paroxysmal hemicrania (PH), and short-lasting unilateral neuralgiform headache attacks with conjunctival injection and tearing (SUNCT). Other examples of disorders or conditions which can be treated according to the present invention include vascular headaches (e.g., migraine headaches), tension headaches, headaches associated with the use of a substance (e.g., triptans such as sumatriptan, benzodiazepines such as alprazolam, analgesics such as ibuprofen, ergots such as ergotamine, opioids such as morphine, recreational drugs such as caffeine, nicotine, alcohol, and hormone replacement therapy containing, for example, estrogen) or its withdrawal. Yet additional examples of disorders or conditions associated with cephalic pain include miscellaneous headache unassociated with a structural lesion, headache associated with a nonvascular intracranial disorder, headache associated with a non-cephalic infection, headache associated with a metabolic disorder, headache associated with a disorder of the cranium, neck, eyes, nose, sinuses, teeth, mouth, or other facial or cranial structure, nerve trunk pain and deafferentation pain.

### Combination Therapies

Optionally the methods for treating a disease or condition described herein can include administering to a patient an intravenous infusion of the psilocin benzoate formulation in combination with one or more additional therapeutic agents, including an antiemetic agent, a benzodiazepine, and anti-inflammatory agents. Additionally, described herein specifically are methods of treating a disease or condition where the disease or condition is chronic pain. When the patient is being treated for chronic pain with the psilocin benzoate formulation the patient may also be administered one or more medications for pain relief, including morphine, hydromorphone, hydrocodone, meperidine, and fentanyl

### Benzodiazepine

In some embodiments described herein, the intravenous psilocin benzoate formulations may be administered to a patient having a psychological condition or a neurological injury in need of treatment in combination with a pharmacologically effective amount of a benzodiazepine. The benzodiazepine may be formulated in the same composition as the psilocin benzoate, or the benzodiazepine may be formulated in a separate compositon from the psilocin benzoate formulations. In some embodiments, the benzodiazepine and the psilocin benzoate formulation are administered concurrently. In certain embodiments, the psilocin benzoate formulation and the benzodiazepine are administered separately.

The benzodiazepine may be a 1,4-benzodiazepine, 1,5-benzodiazepine, 2,3-benzodiazepine, triazolobenzodiazepine, imidazobenzodiazepine, oxazolobenzodiazepine, thienodiazepine, thienotriazolodiazepine, thienobenzodiazepine, pyridodiazepine, pyridotriazolodiazepine, pyrralodiazepine, tetrahydroisoquinobenzodiazepine, a benzodiazepine prodrug, diazepam, midazolam, alprazolam, temazepam, clonazepam, or a combination thereof. In some embodiments, the benzodiazepine is lorazepam.

Lorazepam is a benzodiazepine medication sold under various trade names including ATIVAN®, ALMAZINE®, and TAVOR®. Lorazepam has various properties, including acting as a sedative, a hypnotic, an amnesiac, and an anxiolytic. Like other benzodiazepines, lorazepam is generally understood to act primarily by enhancing the effect of gamma-aminobutyric acid (GABA) at the GABAA receptor. As the primary inhibitory neurotransmitter, GABA acts to reduce neuronal excitability throughout the nervous system. Compared to other benzodiazepines such as diazepam (valium), lorazepam is substantially more potent and longer acting. Lorazepam has the following molecular structure:

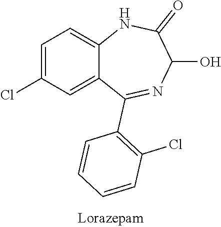 [SMILES: O=C1Nc2ccc(Cl)cc2C(c2ccccc2Cl)=NC1O]

It may be seen, however, that other benzodiazepines may be utilized. In some embodiments described herein, the benzodiazepine is administered in combination with the psilocin benzoate formulation such that the ratio of psilocin benzoate to benzodiazepine is between 100:1 and 10:1 by weight (e.g., 90:1, 80:1, 70:1, 60:1, 50:1, 40:1, 30:1, 20:1 and 10:1 by weight). The benzodiazepine may be administered in a dosage of between 2 mg and 10 mg (e.g., 2±1 mg, 3±1 mg, 4±1 mg, 5±1 mg, 6±1 mg, 7±1 mg, 8±1, 9±1 mg, and 10±1 mg) in combination with the psilocin benzoate formulations. The benzodiazepine administered may be lorazepam. The lorazepam may be administered in a dosage between 2 mg and 4 mg (e.g., 2±1 mg, 3±1 mg, and 4±1 mg). The benzodiazepine administered in combination with the psilocin benzoate formulation may be diazepam. The diazepam may be administered in a dosage between 5 mg and 10 mg (e.g., 5±1 mg, 6±1 mg, 7±1 mg, 8±1, 9±1 mg, and 10±1 mg). The benzodiazepine may be administered orally, transmucosally (e.g. nasally, buccally, sublingually, vaginally, ocularly, rectally, etc.) intravenously, by inhalation, intramuscular injection, and any other form of delivery

The benzodiazepine may be administered to the patient to dampen anxiety-producing effects of the psilocin benzoate. As a result, the benzodiazepine may decrease the intensity of the experience of the subject being administered the psilocin benzoate. Therefore, benzodiazepine may be administered in order to limit, stop, or prevent any negative side effects (such as psilocin-induced anxiety) from the psilocin benzoate that the patient may experience.

### Antiemetic Agent

In some embodiments described herein, the intravenous psilocin benzoate formulation may be administered to a patient having a disease or condition in need of treatment in combination with a pharmacologically effective amount of an antiemetic agent. The antiemetic agent may be administered to the subject prior to the psilocin benzoate formulations. The antiemetic agent may be a non-selective 5-HT antagonist, 5-HT3 receptor antagonist, 5-HT4 receptor agonist, CB1 agonist, D2 receptor antagonist, D3 receptor antagonist, GABA receptor agonist, H1 receptor antagonist, muscarinic acetylcholine receptor antagonist, NK1 receptor antagonist, or a combination thereof. In a preferred embodiment, the antiemetic agent is ondansetron.

Ondansetron is an antiemetic medication sold under the trade names ZOFRAN® and ONDISSOLVE® in various markets. Ondansetron is a 5-HT3 receptor antagonist (a “setron”) generally used in controlling nausea and vomiting in post-operative conditions and in chemotherapy patients, and as well as for various off-label uses. 5-HT3 receptor antagonists bind to and block the 5-HT3 receptor, which is a ligand-gated ion channel found in the vagus nerve and in the area postrema, as well as the in the vomiting center in the medulla oblongata of the brainstem. Synaptic transmission initiated via the 5-HT3 receptor directly mediates the nausea and vomiting reflex. Ondansetron has the following molecular structure:

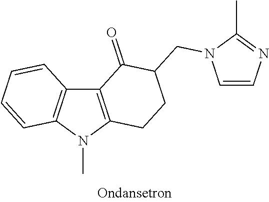 [SMILES: Cc1nccn1CC1CCc2c(c3ccccc3n2C)C1=O]

According to a presently contemplated method of treating a patient, a pharmacologically effective amount of an antiemetic agent may be administered to the patient. The antiemetic agent may be ondansetron. The ondansetron may be administered in a dosage between 4 mg and 8 mg. In some embodiments, the antiemetic agent is administered as a 4 mg intravenous infusion of ondansetron prior to the psilocin benzoate formulations. However, it may be seen that in other embodiments, other preparations may be administered to the patient, which may vary in dosage form. For example, methods of delivery of the antiemetic agent other than intravenous infusion may be utilized, including but not limited to oral delivery, transmucosal (e.g. nasal, buccal, sublingual, vaginal, ocular, rectal, etc.) delivery, inhalatory delivery, intramuscular injection, and any other form of delivery that may achieve administration to the patient of a pharmacologically effective amount of the antiemetic agent. Likewise, it may also be seen that antiemetic agents other than ondansetron may be utilized, including but not limited to other setrons, or other antiemetic compounds or preparations.

### Anti-Inflammatory Agents

In some embodiments described herein, the intravenous psilocin benzoate formulation may be administered to a patient having a disease or condition in need of treatment in combination with a pharmacologically effective amount of anti-inflammatory agent. The anti-inflammatory agent may be formulated in the same composition as the psilocin benzoate formulations, or the anti-inflammatory agent may be formulated in a separate compositon from the psilocin benzoate formulations. In some embodiments, the anti-inflammatory agent and the psilocin benzoate formulation are administered concurrently. In some embodiments, the psilocin benzoate formulation and the anti-inflammatory agent are administered separately. The anti-inflammatory agent may be naproxen, ketoprofen, ibuprofen, tolmetin, etodolac, fenoprofen, diclofenac, flurbiprofen misoprostrol, indomethacin, sulindac, ketorolac, esomeprazole, famotidine, diflunisal, or sumatriptan. In some embodiments, the anti-inflammatory agent is a triptan. In some embodiments, the anti-inflammatory agent is sumatriptan. In some embodiments, the sumatriptan is administered in a dosage of between about 4 mg and about 6 mg (e.g., 4.5 mg, 5.0 mg. 5.5 mg, and 6.0 mg). In particular embodiments, the anti-inflammatory agent is ketorolac. The ketorolc may be administered with a dosage of between about 15 mg and about 30 mg (e.g., 16 mg, 18 mg, 20 mg, 22 mg, 24, mg, 26 mg, 28 mg, and 30 mg). The anti-inflammatory agent may be administered orally, transmucosally (e.g. nasally, buccally, sublingually, vaginally, ocularly, rectally, etc.) intravenously, by inhalation, intramuscular injection, or by another method of delivery.

### OTHER EMBODIMENTS

All publications, patents, and patent applications mentioned in this specification are herein incorporated by reference to the same extent as if each independent publication or patent application was specifically and individually indicated to be incorporated by reference.

Although the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims. Other embodiments are within the claims.

### EXAMPLES

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1: Psilocin Benzoate Formulation for Infusion.

Psilocin as free base was found to be highly unstable in solution (being prone to oxidation) but was assessed to more stable in its benzoate salt form.

### Solubility Testing

Solubility of the psilocin benzoate salt was assessed in a range of potential intravenous vehicles. Vehicles assessed were saline, 5% dextrose, 5% mannitol, 100 mM phosphate buffer pH 5, 100 mM citrate buffer pH 5, 100 mM acetate buffer pH 5. Each vehicle was degassed by sonication prior to use. The results are provided in Table 1.

TABLE 1
Benzoate Salt Solubility Assessment

| | Assay result mg/mL | | |
|---|---|---|---|
| Formulation Vehicle | Preparation | 6 hours | pH |
| Saline | 4.04 | 3.94 | 6.65 |
| 5% Dextrose | 3.56 | 3.48 | 6.67 |
| 5% Mannitol | 3.57 | 3.55 | 6.34 |
| Phosphate buffer pH 5 | 3.78 | 3.80 | 5.62 |
| Acetate buffer pH 5 | 3.70 | 3.88 | 5.59 |
| Citrate buffer pH 5 | 3.98 | 4.02 | 5.54 |

The benzoate salt was partially soluble under saturating conditions in all of the vehicles on test and in each vehicle formed a thin white suspension under saturating conditions. The undissolved drug substance was removed by filtration (0.2 μm PVDF syringe filter) leaving a clear colorless solution. The pH of each formulation was measured and samples for assay analysis from each formulation were taken following preparation and then again following 6 hours bench top storage at ambient temperature. Assay results confirm that in aqueous solution, the limit of solubility of this salt form is approximately 4 mg/ml (3.56 mg/mL to 4.04 mg/mL). Following 6 hours bench top storage, formulations were analyzed again with minimal changes in achieved concentration. No precipitate was observed in any of the solutions. After sampling at the 6-hour time-point the formulations remained on the laboratory bench where it was noted that over time they took on a clear green color (indicative of psilocin degradation) which progressively became more intense, having initially been clear colorless solutions.

### Stability Assessments in Various Buffers

A psilocin benzoate sample stock was prepared at 0.27 mg/ml of the psilocin free base by weighing out about 8.6 mg of the benzoate salt and dissolving this in 20 ml of media (25 mM citrate, 25 mM phosphate, and 25 mM borate buffers) in a volumetric flask. After dissolution, the pH was taken and adjusted to the desired pH where necessary to make buffered solutions having a pH between 3.5 to 5.5. The stocks, and a media ‘blank’, were then studied for stability at 40° C. and were covered in foil to protect the samples from light. A purity analysis was performed at T=0 hours, 24 hours, 48 hours and 1 week of storage, by removing an aliquot from the stock solutions (Table 2). The appearance and pH of each sample was also noted at these times (Table 15 and 16). The purity analysis was performed using an in house high-resolution generic purity method on an Agilent Infinity II series HPLC.

TABLE 2
Purity of psilocin benzoate solutions over time

| | | T = 24 | T = 48 | T = 1 |
|---|---|---|---|---|
| Media | T = 0 | hours | hours | week |
| pH 3.5 Citrate buffer | 99.2 | 98.9 | 98.6 | 95.2* |
| pH 4.0 Citrate buffer | 99.2 | 991. | 98.7 | 94.4* |
| pH 4.5 Citrate buffer | 99.2 | 99.1 | 98.6 | 93.1* |
| pH 5.0 Citrate buffer | 99.1 | 98.9 | 98.0 | 89.5* |
| pH 5.5 Citrate buffer | 99.1 | 98.8 | 97.6 | 83.5* |
| pH 3.5 Phosphate buffer | 99.2 | 97.1 | 95.0 | 89.3* |
| pH 4.0 Phosphate buffer | 99.2 | 96.3 | 93.7 | 86.1* |
| pH 4.5 Phosphate buffer | 99.2 | 97.3 | 94.3* | 83.3* |
| pH 5.0 Phosphate buffer | 99.2 | 97.4 | 93.8* | 76.5* |
| pH 5.5 Phosphate buffer | 99.2 | 97.9 | 95.5* | 79.1* |
| pH 3.5 Borate buffer | 99.1 | 98.0 | 97.6 | 91.7* |
| pH 4.0 Borate buffer | 99.1 | 96.7* | 94.5* | 74.0* |
| pH 4.5 Borate buffer | 99.2 | 98.0 | 95.1* | 65.3** |
| pH 5.0 Borate buffer | 99.2 | 98.1* | 95.0* | 73.6* |
| pH 5.5 Borate buffer | 99.2 | 96.7* | 95.9* | 77.7* |

*Chromatography shows a large broad peak which made integration difficult

The pH 3.5 citrate, pH 4.0 citrate, and pH 4.5 citrate solutions maintained the desired pH and had the greatest purity of 95.2%, 94.4%, and 93.1%, respectively, at the 1-week time point. These samples were also the last to exhibit a change in color, which occurred between the 48 hour and 1 week time point. In contrast, phosphate and borate buffers exhibited significantly more degradation at the 1-week time point.

### Assessment of Citrate and Acetate Formulation Buffers

The effect of buffer on the stability of the formulation was assessed using comparative formulations (prepared at pH 4, 4.5, and 5), with all buffer concentrations set at 100 mM. Results in terms of appearance and assay for psilocin benzoate are tabulated below (Tables 3 and 4). Formulations were assessed at preparation, following 6 hours bench top storage, and then again after 24 hours bench top storage.

TABLE 3
Benzoate Salt, Formulation Appearance

Appearance

| Vehicle | Preparation | 6 hours | 24 hours |
|---|---|---|---|
| Citrate pH 4 | clear, | clear, | pale green |
| | colourless | colourless | solution |
| Citrate pH 4.5 | clear, | clear, | pale green |
| | colourless | colourless | solution |
| Citrate pH 5 | clear, | clear, | pale green |
| | colourless | colourless | solution |
| Acetate pH 4 | clear, | clear, | dark green |
| | colourless | colourless | solution |
| Acetate pH 4.5 | clear, | colour hint | dark green |
| | colourless | | solution |
| Acetate pH 5 | clear, | colour hint | dark green |
| | colourless | | solution |

TABLE 4
Benzoate Salt Assay

| | Assay mg/mL | | | Change |
|---|---|---|---|---|
| Vehicle | Preparation | 6 hours | 24 hours | (%) |
| Citrate pH 4 | 2.95 | 2.91 | 2.92 | −1.4 |
| Citrate pH 4.5 | 2.94 | 2.93 | 2.92 | −0.7 |
| Citrate pH 5 | 3.07 | 3.09 | 3.05 | −0.7 |
| Acetate pH 4 | 3.03 | 3.04 | 2.99 | −1.3 |
| Acetate pH 4.5 | 3.04 | 3.04 | 2.97 | −2.3 |
| Acetate pH 5 | 3.07 | 3.05 | 2.98 | −2.9 |

As with the solubility assessment, the formulations became more intensely colored (degraded) as they aged. The psilocin benzoate exhibited a significant color distinction between the citrate buffer (at all pH's) and the acetate buffer, where the acetate buffer solutions took on color more quickly and more intensely. All formulations were accurately prepared and losses in concentration over 24 hours storage suggest that losses are reduced using a citrate buffer system rather than an acetate buffer system. The impact of pH on formulations in both buffers again is only marginal with no clear trend. Based upon these observations, the use of citrate as a buffer is superior over acetate as a buffer, with the most marked difference being that the citrate formulations took on color at a slower rate than the acetate formulations.

### Assessment of Formulation Antioxidants

To reduce the oxidation of the psilocin benzoate solution, an antioxidant excipient (ascorbic acid) was assessed as an additional excipient with the addition of ascorbic acid at a final concentration of 1% w/v or 10 mg/mL. Formulations were prepared at 3 mg/mL and 0.3 mg/mL, aliquoted and analyzed by appearance and HPLC over time and storage. Formulations were stored in clear glass bottles that either sat on the bench top at ambient temperature or were refrigerated.

The assay results remained within 3.5% of the initial concentration at all time-points. In terms of appearance, color was observed at 24 hours in the 3 mg/mL formulation (ambient storage) and intensified to a distinct yellow color by 96 hours. Due to the lower concentration of the 0.3 mg/mL formulation color was only truly observed in the 96 hour ambient sample. The refrigerated formulations however remained clear at 24 hours and even up to 96 hours. The straw yellow color observed in this experiment was seen for the first time and hence is likely linked to the addition of the ascorbic acid. There was a progressive increase in related substances (HPLC RRT 0.74) at both test concentrations and was most prominent in the ambient stored formulations, and pH remained stable throughout with small fluctuations in osmolality.

Changes in formulation appearance (when stored at ambient in clear glass containers on the bench) are consistent with previously prepared formulations without the addition of ascorbic acid. Changes in appearance and an increase in related substances was substantially retarded in the refrigerated stored formulations.

### Stability Assessments Over Time Using Various Antioxidants

A psilocin benzoate stock was prepared at a concentration of 0.27 mg/ml of the psilocin free base form by weighing out about 6.45 mg of the benzoate salt and dissolving it in 15 mL of media to make the solutions described in Table 5. Aliquots were taken from the stock to prepare individual vials for each time point at each temperature. Samples were wrapped in foil to protect from light and stored at the following temperatures: 2-8° C. (Refrigerated), and 25° C.

TABLE 5
Psilocin benzoate solutions tested

| Media | pH |
|---|---|
| pH 4.5 Citrate buffer in 0.45% Saline | 4.7 |
| 0.05% Sodium Bisulfite in pH 4.5 Citrate buffer in | 4.6 |
| 0.45% Saline | |
| 1% Sodium Metabisulfite in pH 4.5 Citrate buffer in | 4.4 |
| 0.45% Saline | |
| 1% Ascorbate in pH 4.5 Citrate buffer in 0.45% | 4.6 |
| Saline | |
| 0.2% Sodium sulfite in pH 4.5 Citrate buffer in | 4.6 |
| 0.45% Saline | |
| 0.5% Thioglycerol in pH 4.5 Citrate buffer in | 4.6 |
| 0.45% Saline | |

Purity analysis was performed at T=0, 2, 4, 8, 24 and 48 hours and then after 5 days of storage. The appearance and pH of each sample were also noted. After analysis, each sample was placed in the freezer for storage. The purity analysis was performed using an in house high resolution generic purity method on an Agilent Infinity II series HPLC. A content assay was also performed in order to quantify the amount of free base psilocin remaining in solution. The purity results over time were measured (Table 6).

TABLE 6
Purity of psilocin benzoate solutions over time

| 25° C. | | | | | | | | 5° C. |
|---|---|---|---|---|---|---|---|---|
| | Area Purity (%) | | | | | | | |
| | | T = 2 | T = 4 | T = 8 | T = 24 | T = 48 | 5 | 5 |
| Media | T = 0 | Hours | Hours | Hours | Hours* | Hours* | days* | days* |
| Citrate Buffer | 99.2 | 98.3 | 98.5 | 98.4 | 93.5 | 98.6 | 91.3 | 97.6 |
| (Control) | | | | | | | | |
| 0.05% Sodium | 95.0 | 95.1 | 94.5 | 94.6 | 94.0 | 96.5 | 95.1 | 97.7 |
| Bisulfite in buffer | | | | | | | | |
| 0.2% Sodium | 97.0 | 96.4 | 96.2 | 96.1 | 96.8 | 96.9 | 87.5 | 97.7 |
| Sulfite in buffer | | | | | | | | |
| 0.5% Thioglycerol | 98.5 | 98.4 | 98.4 | 97.5 | 98.6 | 98.9 | 98.5 | 98.2 |
| in buffer | | | | | | | | |
| 1% Ascorbate | 97.4 | 96.0 | 95.3 | 93.1 | 81.5 | 77.8 | 33.6 | 83.0 |
| in buffer | | | | | | | | |

*Column degradation and increased pressure observed on the system, even after multiple column regenerations.

### Stability at Various Temperatures and Light Exposure Using Various Antioxidants

A sample stock solution of psilocin benzoate was prepared at a concentration of 0.27 mg/ml of the free base of psilocin by weighing out about 43 mg of the benzoate salt and dissolving this in 100 ml of media in a volumetric flask to generate the solutions described in Table 7. The stocks, and a media ‘blank’, were then studied for stability at the following conditions:
2-8° C. (Refrigerated)-Dark
25° C.—Exposed to light
25° C.—Not exposed to light
40° C.—Exposed to light
40° C.—Not exposed to light

TABLE 7
Psilocin benzoate solutions tested

| Media | pH |
|---|---|
| pH 4.5 Citrate buffer in 0.45% Saline | 4.5 |
| 0.01% Sodium Bisulfite in pH 4.5 Citrate buffer in | 4.5 |
| 0.45% Saline | |
| 0.03% Sodium Bisulfite in pH 4.5 Citrate buffer in | 4.5 |
| 0.45% Saline | |
| 0.05% Sodium Bisulfite in pH 4.5 Citrate buffer in | 4.5 |
| 0.45% Saline | |
| 0.1% Thioglycerol in pH 4.5 Citrate buffer in | 4.5 |
| 0.45% Saline | |

Purity analysis was performed at T=0 hours, 24 hours, 48 hours, 1 week and 2 weeks of storage, by removing an aliquot from the stock solutions (Table 8). The appearance and pH of each sample was also noted at each timepoint. After analysis, the sample and ‘blank’ aliquots were placed in the freezer for storage. The purity analysis was performed using an house high-resolution generic purity method on an Agilent Infinity II series HPLC. A content assay was also performed in order to quantify the amount of free base psilocin that remained in solution (Table 9).

TABLE 8
Purity of psilocin benzoate solutions over time

| | | | Purity (%) | | | | | |
|---|---|---|---|---|---|---|---|---|
| | Temperature | Exposed | | T = 24 | T = 48 | T = 1 | T = 2 | T = 4 |
| Media | (° C.) | to light? | T = 0 | Hours | Hours | Week | Weeks | Weeks |
| pH 4.5 | 2-8 | N | 99.1 | 99.2 | 99.1 | 99.0 | 98.7 | 98.4 |
| Citrate | 25 | Y | | 99.2 | 99.1 | 98.8 | 97.6 | 93.8 |
| saline | 25 | N | | 99.2 | 99.2 | 99.0 | 98.1 | 95.0 |
| buffer | 40 | Y | | 99.1 | 98.6 | 90.0 | 58.5* | 19.5* |
| | 40 | N | | 99.0 | 98.5 | 77.9 | 51.1* | 20.5* |
| 0.01% sodium | 2-8 | N | 99.2 | N/A | 98.6 | 98.3 | N/A | N/A |
| bisulfite in | 25 | Y | | | 98.3 | 96.9 | | |
| pH 4.5 Citrate | 25 | N | | | 98.4 | 97.3 | | |
| Saline Buffer | 40 | Y | | | 96.9 | 93.7 | | |
| | 40 | N | | | 96.9 | 94.0 | | |
| 0.03% sodium | 2-8 | N | 99.0 | N/A | 98.3 | 97.5 | N/A | N/A |
| bisulfite in | 25 | Y | | | 97.6 | 94.3 | | |
| pH 4.5 Citrate | 25 | N | | | 97.8 | 95.4 | | |
| Saline Buffer | 40 | Y | | | 94.4 | 88.6 | | |
| | 40 | N | | | 94.7 | 88.6 | | |
| 0.05% sodium | 2-8 | N | 98.8 | 98.8 | 98.5 | 97.7 | 96.4 | 94.1 |
| bisulfite in | 25 | Y | | 98.3 | 97.2 | 92.4 | 86.5 | 80.5 |
| pH 4.5 Citrate | 25 | N | | 98.4 | 97.8 | 95.5 | 92.6 | 88.8 |
| Saline Buffer | 40 | Y | | 96.6 | 94.0 | 87.0 | 81.0 | 65.3 |
| | 40 | N | | 96.6 | 93.9 | 86.8 | 81.4 | 75.0 |
| 0.1% | 2-8 | N | 99.2 | 99.3 | 99.2 | 99.2 | 98.9 | 98.9 |
| Thioglycerol | 25 | Y | | 99.2 | 99.2 | 98.6 | 97.8 | 94.9 |
| in pH 4.5 | 25 | N | | 99.2 | 99.2 | 98.7 | 97.9 | 95.1 |
| Citrate | 40 | Y | | 98.8 | 98.3 | 94.1 | 74.0 | 8.5* |
| saline buffer | 40 | N | | 98.7 | 98.2 | 88.0 | 52.4 | 9.3* |

*Large broad peak observed in the chromatograph

TABLE 9
Stability of Psilocin Free base in Solution

| | | Exposed | Content (mg/mL) | | | |
|---|---|---|---|---|---|---|
| | Temperature | to | | T = 24 | T = 48 | T = 1 |
| Media | (° C.) | light? | T = 0 | Hours | Hours | Week |
| 0.01% | 2-8 | N | 0.27 | N/A | 0.30 | 0.26 |
| sodium | 25 | Y | 0.27 | | 0.26 | 0.25 |
| bisulfite | 25 | N | 0.27 | | 0.26 | 0.25 |
| in pH 4.5 | 40 | Y | 0.27 | | 0.26 | 0.24 |
| Citrate | 40 | N | 0.27 | | 0.26 | 0.24 |
| Saline | | | | | | |
| Buffer | | | | | | |
| 0.03% | 2-8 | N | 0.27 | N/A | 0.26 | 0.25 |
| sodium | 25 | Y | 0.27 | | 0.26 | 0.25 |
| bisulfite | 25 | N | 0.27 | | 0.26 | 0.25 |
| in pH | 40 | Y | 0.27 | | 0.26 | 0.24 |
| 4.5 | 40 | N | 0.27 | | 0.26 | 0.24 |
| Citrate | | | | | | |
| Saline | | | | | | |
| Buffer | | | | | | |
| 0.05% | 2-8 | N | 0.28 | 0.27 | 0.27 | 0.26 |
| sodium | 25 | Y | 0.28 | 0.26 | 0.26 | 0.26 |
| bisulfite | 25 | N | 0.28 | 0.27 | 0.27 | 0.26 |
| in pH | 40 | Y | 0.28 | 0.26 | 0.26 | 0.25 |
| 4.5 | 40 | N | 0.28 | 0.26 | 0.26 | 0.25 |
| Citrate | | | | | | |
| Saline | | | | | | |
| Buffer | | | | | | |

The 0.01% thioglycerol samples showed comparable purity and content to the citrate buffer at 40° C. at the 4-week time point. At the lower temperatures, however, the thioglycerol samples showed improved stability when compared to the citrate buffer. At the 4-week time point the purity of samples in 0.05% sodium bisulfite, which were exposed to light, were significantly reduced when compared to samples not exposed to light.

When assessing the varying concentrations of sodium bisulfite, the following was observed:
No change in color over 1 week for all concentrations of sodium bisulfite,
Concentrations of 0.01% sodium bisulfite had greater purity at the 1 week time point when compared to the 0.03% and 0.05% and the citrate buffer,
At the 1-week time point, the 0.01% sodium bisulfite samples, under all conditions, had a purity of greater than 93%, and exhibited superior stability when exposed to higher temperatures.

### Example 2: Lyophilized Psilocin Benzoate Formulation for Reconstitution Prior to Infusion

The psilocin benzoate pharmaceutical composition can be provided as a lyophilized powder. A psilocin benzoate solution containing citrate buffer and an antioxidant is freeze dried to produce a lyophilized powder that can be reconstituted in water or the appropriate vehicle prior to infusion into a subject. For example, the lyophilized powder containing between 0.16 mg and 20 mg of psilocin benzoate, citrate buffer, sodium bisulfite, and sodium chloride can be dissolved in 30 ml of water to produce a reconstituted solution for injection. The sodium chloride may be in the lyophilized powder or a saline solution having 0.4% (w/v) sodium chloride may be used. An anti-caking agent, such as mannitol may also be included.

The lyophilized powder is then reconstituted with normal saline to produce a solution including between 5.33 μg/mL and 667 μg/mL psilocin benzoate, 1 mg/mL sodium bisulfite, 15.8 mg/mL citric acid monohydrate, 22.1 mg/mL. citric acid trisodium dihydrate, and 4 mg/mL sodium chloride.

The reconstituted solution includes 150 mM citrate, 0.4% (w/v) sodium chloride, 0.01% (w/v) sodium bisulfite, and a pH of 4.5.

### Example 3: Evaluation of the Safety and Efficacy of Intravenous Infusion of Psilocin Benzoate for Patients Having Depression.

Subjects suffering from depression are treated with an intravenous psilocin benzoate formulation.

To enhance participant safety, all subjects undergo: 1) a preparation session with a staff member prior to dosing; 2) administration of study medications in an aesthetically pleasing room under the supervision of one attendant with video/audio monitoring by a remote staff member; and 3) two post-dose integration sessions during which participants are encouraged to discuss their intervention experience with the attendant.

On Day 1, patients are screened to ensure they are between the ages of 25 to 75 years of age, have sustained moderate severe depression symptoms, meet the Diagnostic and Statistical Manual of Mental Disorders-5 criteria for a diagnosis of major depressive disorder and are currently experiencing a major depressive episode of at least a 60-day duration at the time of screening, are able to provide informed consent, are able to stop standard of care treatment for a wash-out period, and have an identified support person and agree to be accompanied home by that person following dosing. Upon selection from the screening, the baseline depression for each patient is evaluated using the Montgomery-Asberg Depression Rating Scale (MADRS) along with other pre-drug baseline assessments. Psilocin benzoate is then administered intravenously to the patient in a dose infusion rate of between 6.4 mg/hr and 12.8 mg/hr over a period of up to 60 minutes, and a Cmax per dosing of about 30 ng/ml.

While the subject is being administered the psilocin benzoate infusion, the subject's intensity of experience of the psilocin benzoate is monitored using the Drug Effects Questionnaire (DEQ). The infusion rate of psilocin benzoate is reduced when the subject experiences any effects that the subject deems uncomfortable, including confusion, paranoia, or hallucinations, resulting in a decrease in the subject's intensity of experience of the psilocin benzoate. The subject's plasma concentration of psilocin is also monitored while the psilocin benzoate infusion in being administered.

Following administration of the psilocin benzoate, a post-drug assessment is performed. On Day 2, the patient attends a telemedicine appointment for integration and support, and assessments are performed via an online platform. On Day 8, the patient attends a telemedicine appointment for integration and support; assessments are performed via an online platform, and a MADRS-C evaluation is conducted by a third-party rater. On Day 30, the patient attends a telemedicine appointment for integration and support; assessments are performed via an online platform, and a MADRS-C evaluation is conducted by a third-party rater.

Primary outcome measures are the score of depression severity as measured by the MADRS-C 1 week and 4 weeks after receiving treatment. Secondary outcome measures are a sustained depressive symptom response defined as a ≥50% reduction from Baseline MADRS score at all post-dose assessments, sustained depressive symptom remission defined as a central rater MADRS total score of ≤10 at all post-dose assessments, and an evaluation of the effects of psilocin benzoate therapy on the patient quality of life (EQ-5D-3L), anxiety (GAD-7), PHQ-9, positive effect, and stress scale. Patients are also evaluated for changes in serum biomarkers of inflammation, specifically: Tumor Necrosis Factor-Alpha (TNF-a), interleukin (IL)-6, IL-10, IL-1β, C-reactive Protein (CRP), and Macrophage Migration Inhibitory Factor (MIF) at baseline and various timepoints. Patients are also evaluated for changes in biomarkers of metabolic and cardiovascular function, specifically HDL cholesterol, LDL cholesterol, triglycerides, fasting blood glucose, insulin, blood pressure (systolic/diastolic), at baseline, and various timepoints

Patients are evaluated for a variety of psychometrics related to mood, affect, and outlook, including trait and state predictors of dose response. Patients are evaluated for dose response in relation to atypical vs. melancholic features presented by the patient (i.e. BMI, interpersonal (rejection) sensitivity, rumination, appetite, hyper/hyposomnia, leaden paralysis, etc.) as evaluated by [TBD] depressive subtype analysis. Lastly, patients are evaluated by ambulatory assessments and digital biomarkers via mobile health adjuncts.

The psilocin benzoate infusions can reduce symptoms of depression in subjects suffering from depression or a condition associated with depression.

### Example 4: Treatment of Patients Having Anxiety with Intravenous Infusion of Psilocin Benzoate

Subjects suffering from anxiety are treated with an intravenous infusion of a pharmaceutical composition including psilocin benzoate. The subject is first diagnosed with anxiety by a clinician by using a physical exam, using the criteria listed in the Diagnostic and Statistical Manual of Mental Disorders (DSM-5), or conducting an anxiety screening test such as the Zung Self-Rating Anxiety Scale, the Hamilton Anxiety Scale, the Beck Anxiety Inventory, the Social Phobia Inventory, the Penn State Worry Questionnaire, the Yale-Brown Obsessive-Compulsive Scale, or the General Anxiety Disorder-7. The subjects are administered a continuous intravenous dosage of the pharmaceutical composition including psilocin benzoate with an infusion having between 1.6 mg and 24 mg of psilocin benzoate over a time period of no more than one hour. The subject is administered a dosage of the pharmaceutical composition including psilocin benzoate up to 2 and 3 times a week for a period of 4 weeks. After 4 weeks, the subject's symptoms associated with anxiety are evaluated using the criteria listed in the Diagnostic and Statistical Manual of Mental Disorders (DSM-5), or the score received on the Zung Self-Rating Anxiety Scale, the Hamilton Anxiety Scale, the Beck Anxiety Inventory, the Social Phobia Inventory, the Penn State Worry Questionnaire, the Yale-Brown Obsessive-Compulsive Scale, or the Patient Health Questionnaire-9. The psilocin benzoate formulation can reduce anxiety in subjects suffering from anxiety or a condition associated with anxiety.

### Example 5: Treatment of Patients Having a Psychosomatic Condition with Intravenous Infusion of Psilocin Benzoate

Subjects suffering from a somatic disorder experiencing a psychosomatic condition are treated with an intravenous infusion of a pharmaceutical composition including psilocin benzoate. The subject is first diagnosed by a clinician with somatic disorder as experiencing a psychosomatic condition including fibromyalgia, back pain, migraine, and chronic fatigue syndrome after having first received a physical examination to rule out physical causes for the symptoms experienced by the subject. The subject is also evaluated using the criteria listed in the Diagnostic and Statistical Manual for Mental Disorders (DSM-5) for a somatic disorder. The subject is administered a continuous intravenous dosage of the pharmaceutical composition including psilocin benzoate having between 1.6 mg and 24 mg of psilocin benzoate over a time period of no more than an hour. The subject is administered the intravenous infusion of psilocin benzoate 2 times a week for a period of 3 weeks. The subject's psychosomatic symptoms are then evaluated by a clinician in terms of intensity and frequency of the symptoms that the subject is experiencing currently compared to before receiving treatment. The psilocin benzoate infusions can reduce the intensity and frequency of psychosomatic symptoms in subjects suffering from a psychosomatic condition.

### Example 6: Treatment of Patients Having Post-Traumatic Stress Disorder with Intravenous Infusion of Psilocin Benzoate

Subjects suffering from post-traumatic stress disorder (PTSD) are treated with an intravenous infusion of the pharmaceutical composition including psilocin benzoate. The subject is first diagnosed by a clinician with post-traumatic stress disorder using a physical exam and the criteria listed in the Diagnostic and Statistical Manual for Mental Disorders (DSM-5). The subjects are administered a continuous intravenous dosage of the pharmaceutical composition including psilocin benzoate having between 1.6 mg and 24 mg of psilocin benzoate over a time period of no more than an hour. The subjects are simultaneously administered the benzodiazepine lorazepam in the same intravenous formulation as the psilocin benzoate formulation in a dosage of 2 mg to 4 mg. The subject is administered the intravenous infusion of the pharmaceutical composition including psilocin benzoate and lorazepam up to 3 times a week for one week.

One week after the first administration of the pharmaceutical composition including the psilocin benzoate, the subject's symptoms associated with post-traumatic stress disorder are evaluated by a clinician using the criteria listed in the DSM-5. In particular, the subject's anxiety level is assessed using an anxiety screening test such as the Zung Self-Rating Anxiety Scale, the Hamilton Anxiety Scale, the Beck Anxiety Inventory, or the General Anxiety Disorder-7. Additionally, as a result of receiving treatment, the subject is capable of adaptive reconsolidation of the subject's traumatic memory, resulting in a reduction in anxiety, depression, aggression, and/or hypervigilance. As a result of the administration of the psilocin benzoate formulation in combination with lorazepam, the subject can experience less anxiety in comparison the amount of anxiety experienced before treatment. Based on the subject's symptoms associated with post-traumatic stress disorder after receiving treatment in comparison to before receiving treatment, the clinician may recommend continued treatment. The psilocin benzoate formulation can reduce the intensity and frequency of PTSD symptoms (e.g., anxiety or depression) in subjects suffering from PTSD.

### Example 7: Treatment of Patients Having a Traumatic Brain Injury with Intravenous Infusion of Psilocin Benzoate

Subjects suffering from a traumatic brain injury are treated with an intravenous infusion of a pharmaceutical composition including psilocin benzoate. The subjects are first diagnosed by a clinician with a traumatic brain injury using a physical exam. The subjects are administered an intravenous dosage of the pharmaceutical composition including psilocin benzoate having between 1.6 mg and 20 mg of psilocin benzoate over a time period of 30 minutes to 4 hours. The subject is administered the intravenous infusion of the pharmaceutical composition including psilocin benzoate up to 3 times a week for 4 weeks. After four weeks, the subject is evaluated by a clinician using a physical exam and brain scans in order to evaluate the injury and acute inflammation.

### Example 8: Treatment of Patients Having a Spinal Cord Injury with Intravenous Infusion of Psilocin Benzoate

Subjects suffering from a spinal cord injury are treated with an intravenous infusion of a pharmaceutical composition including psilocin benzoate. The subject is first diagnosed with a spinal cord injury by a physical exam including an MRI or CT scan. The subject is administered an intravenous dosage of the pharmaceutical composition including psilocin benzoate having between 1.6 mg and 24 mg of psilocin benzoate over a time period of between 30 minutes and 4 hours, 3 times a week for a period of 4 weeks. After 4 weeks, the subject is reevaluated by clinician including a physical exam to identify any physical improvements resulting from receiving treatment. The subject's symptoms associated with the spinal cord injury are evaluated and compared to the subject's symptoms before receiving treatment. The subject's pain before and after treatment is evaluated using a numerical pain scale, a Wong-Baker faces pain scale, a FLACC pain scale, a CRIES pain scale, a COMFORT pain scale, a McGill pain scale, a color analog pain scale, a Mankoski pain scale, a Brief Pain Inventory, or a Descriptor Differential Scale 10 of Pain Intensity. The subject's impairment is rated before and after treatment using the Asia Impairment Scale (AIS) or the International Standards for Neurological Classification of Spinal Cord Injury ISNCSCI. The subject is also evaluated in terms of motor and sensory capabilities before and after receiving treatment. The psilocin benzoate infusions can reduce pain in subjects suffering from a spinal cord injury.

What is claimed is:

1. A pharmaceutical composition comprising (i) an aqueous solution having a pH of between about 3.5 and about 6.5, (ii) between about 0.0053 mg/mL and about 4.0 mg/ml of psilocin benzoate, and (iii) an antioxidant, wherein the pharmaceutical composition is suitable for infusion.

2. The pharmaceutical composition of claim 1, wherein the aqueous solution comprises a citrate buffer, an acetate buffer, or a phosphate buffer, and has a pH of between about 4.0 and about 6.0.

3. A pharmaceutical composition comprising (i) a citrate buffered aqueous solution having a pH of between about 4.0 and about 5.0, and (ii) between about 0.0053 mg/mL and about 4.0 mg/ml of psilocin benzoate, wherein the pharmaceutical composition is suitable for infusion.

4. The pharmaceutical composition of any one of claims 1-3, wherein the aqueous solution comprises between about 0.001% (w/v) to 2% (w/v) of an antioxidant.

5. The pharmaceutical composition of any one of claims 1-4, wherein the antioxidant is vitamin C, thioglycerol, sodium bisulfite, or sodium sulfite.

6. The pharmaceutical composition of claim 5, wherein the aqueous solution comprises about 0.01+0.005% sodium bisulfite.

7. The pharmaceutical composition of any one of claims 1-6, wherein the pharmaceutical composition further comprises one or more pharmaceutically acceptable excipients selected from a preservative, or a tonicity agent.

8. The pharmaceutical composition of claim 7, wherein the pharmaceutical composition further comprises from 0.1% (w/v) to 1% (w/v) sodium chloride as a tonicity agent.

9. The pharmaceutical composition of any one of claims 1-8, wherein the pharmaceutical composition comprises: (i) a citrate buffered aqueous solution having a pH of between 4.0 and 5.0, (ii) between about 0.0053 mg/mL and about 0.7 mg/mL of psilocin benzoate, and (iii) 0.01 to 0.075% (w/v) sodium bisulfite.

10. The pharmaceutical composition of claim 9, wherein the pharmaceutical comprises from about 50 to about 200 mM citrate buffer.

11. A reconstitutable powder comprising psilocin benzoate, an antioxidant, and a buffer, wherein reconstitution of the reconstitutable powder in from 5 mL to 50 ml of an aqueous solution produces the pharmaceutical composition of any one of claims 1-10.

12. The reconstitutable powder of claim 11, wherein the powder comprises between about 0.01% (w/w) and 2% (w/w) of psilocin benzoate.

13. A method of treating a disease or condition in a subject in need thereof, the method comprising intravenously administering to the subject a pharmaceutical composition of any one of claims 1-10 in an amount sufficient to treat the disease or condition.

14. A method of treating a disease or condition in a subject in need thereof, the method comprising intravenously administering to the subject a pharmaceutical composition of any one of claims 1-10 over a period of between 1 minute and 60 minutes.

15. The method of claim 13 or claim 14, wherein the pharmaceutical composition is administered to the subject over a period of 20 to 60 minutes.

16. The method of any one of claims 13-15, wherein the disease or condition is a neurological injury, a neurodegenerative disease, an inflammatory condition, chronic pain, or a psychological condition.

17. The method of claim 16, wherein the disease or condition is an inflammatory condition.

18. The method of claim 17, wherein the inflammatory condition is lung inflammation, neuroinflammation, rheumatoid arthritis, atherosclerosis, psoriasis, type II diabetes, inflammatory bowel disease, Crohn's disease, multiple sclerosis, and/or septicemia.

19. The method of claim 17, wherein the inflammatory condition is chronic obstructive pulmonary disease (COPD), or Alzheimer's disease.

20. The method of claim 16, wherein the disease or condition is a neurological injury.

21. The method of claim 20, wherein the neurological injury is a stroke, a traumatic brain injury, or a spinal cord injury.

22. The method of claim 16, wherein the disease or condition is chronic pain.

23. The method of claim 22, wherein the chronic pain results from post-operative pain, tension headaches, chronic lower back pain, fibromyalgia, nephropathy, multiple sclerosis, shingles, complex regional pain syndrome, cephalic pain, or sciatica.

24. The method of claim 22, wherein the chronic pain condition results from trigeminal autonomic cephalalgia.

25. The method of claim 24, wherein the trigeminal autonomic cephalalgia is selected from the group consisting of episodic and chronic cluster headache (CH), episodic and chronic paroxysmal hemicrania (PH), and short-lasting unilateral neuralgiform headache attacks with conjunctival injection and tearing (SUNCT).

26. The method of claim 25, wherein the trigeminal autonomic cephalalgia is episodic or chronic CH.

27. The method of claim 16, wherein the condition is a psychological condition.

28. The method of claim 27, wherein the psychological condition is depression, anxiety, addiction, post-traumatic stress disorder, an eating disorder, or compulsive behavior.

29. The method of claim 27, wherein the psychological condition is depression.

30. The method of claim 27, wherein the psychological condition is anxiety.

31. The method of claim 16, wherein the disease or condition is a neurodegenerative disease selected from Alzheimer's disease, Huntington's disease, and Parkinson's disease.

32. The method of any one of claims 13-31, wherein the method comprises further administering to the patient a pharmacologically effective amount of an antiemetic agent.

33. The method of claim 32, wherein the antiemetic agent comprises a non-selective 5-HT antagonist, 5-HT3 receptor antagonist, 5-HT4 receptor agonist, CB1 agonist, D2 receptor antagonist, D3 receptor antagonist, GABA receptor agonist, H1 receptor antagonist, muscarinic acetylcholine receptor antagonist, NK1 receptor antagonist, or a combination thereof.

34. The method of claim 32 or claim 33, wherein the antiemetic ondansetron is intravenously infused.

35. The method of any one of claims 13-34, wherein the intravenous infusion comprises a pharmacologically effective amount of a benzodiazepine.

36. The method of claim 35, wherein the benzodiazepine is a 1,4-benzodiazepine, 1,5-benzodiazepine, 2,3-benzodiazepine, triazolobenzodiazepine, imidazobenzodiazepine, oxazolobenzodiazepine, thienodiazepine, thienotriazolodiazepine, thienobenzodiazepine,pyridodiazepine, pyridotriazolodiazepine, pyrralodiazepine, tetrahydroisoquinobenzodiazepine, a benzodiazepine prodrug, or a combination thereof.

37. The method of claims 35 and 36, wherein the benzodiazepine is lorazepam or diazepam.

38. The method of any one of claims 35-37, wherein the benzodiazepine is administered in a dosage between 2 mg and 10 mg.

39. The method of any one of claims 13-38, wherein the intravenous infusion comprises a pharmacologically effective amount of an anesthetic, a sedative, an antiemetic, an anticonvulsant, an antidepressant, an antimigraine, an antipsychotic, an anxiolytic, and/or an antiparkinson agent.

40. The method of any one of claims 13-39, further comprising administering to the patient a preparation comprising a pharmacologically effective amount of an anti-inflammatory agent.

41. The method of claim 40, wherein the administration of the preparation is an intravenous infusion of ketorolac or pharmaceutically acceptable salt thereof.

42. The method of claim 40, wherein the administration of the preparation is an intramuscular infusion of a pharmacologically effective amount of a triptan or a pharmaceutically acceptable salt thereof.

43. The method of claim 40, wherein the preparation comprises sumatriptan or a pharmaceutically acceptable salt thereof.

44. The method of any one of claims 13-43, wherein the intravenous infusion comprising a pharmacologically effective amount of psilocin benzoate is administered at least twice over the course of a month.

45. The method of claim 44, wherein the intravenous infusion comprising a pharmacologically effective amount of psilocin benzoate is administered between 2 and 10 times over the course of a year.
